(19) United States
(12) Patent Application Publication
(10) Pub. No.: US 2009/0149778 A1
(43) Pub. Date: Jun. 11, 2009

(54) DEPRESSION DETECTION SYSTEM

(75) Inventors: Elke Naujokat, Aachen (DE); Micheal Perkuhn, Aachen (DE); Matthew Harris, Aachen (DE); Christine Norra, Aachen (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/719,558

(22) PCT Filed: Nov. 14, 2005
(86) PCT No.: PCT/IB05/53738

§ 371 Date: May 17, 2007

(30) Foreign Application Priority Data
Nov. 23, 2004 (EP) 04106001.3

(51) Int. Cl. A61B 5/11; A61B 5/00

(52) U.S. Cl. 600/595; 600/300

(57) ABSTRACT

The present invention relates to a depression detection system (2). Furthermore the invention relates to a method of detecting depressions and to a computer program. In order to provide an objective measure for a depression as well as for a relapse into depression, a depression detection system is suggested, the system comprising a patient-based device (2) using an actimetric sensor unit (3) adapted to measure a patient's activity and further comprising a signal processing unit (4) adapted to obtain a depression detection result based on the measured activity of the patient.

The present invention relates to a depression detection system. Furthermore, the invention relates to a method of detecting depressions and to a computer program.

Depression is one of the most frequent diseases. It affects approximately one out of five patients in the weeks after an acute myocardial infarction and is associated with an increased risk of cardiac morbidity and mortality. Since depression can have devastating effects on the performance and quality of life not only of the patient him/herself but also of his/her partner and relatives, there is an intense interest in how to achieve an early diagnosis followed by an early treatment. Today, depression is diagnosed by means of detailed patient interviews, including the patient's partner and/or relatives. Standard assessment scales (e.g. Hamilton's depression scale) are used for this. An objective measure for a depression is lacking, however, as is an objective measure for a relapse into depression.

It is an object of the present invention to provide an objective measure for depression as well as for a relapse into depression.

This object is achieved according to the invention by a depression detection system, the system comprising a patient-based device comprising an actimetric sensor unit adapted to measure a patient's activity and a signal-processing unit adapted to obtain a depression detection result based on the measured activity of the patient.

The object of the present invention is also achieved by a method of detecting depression, the method comprising the steps of measuring a patient's activity by means of an actimetric sensor unit, and obtaining a depression detection result based on the measured activity of the patient.

The object of the present invention is also achieved by a computer program comprising computer instructions to measure a patient's activity using an actimetric sensor unit, and computer instructions to obtain a depression detection result based on the measured activity of the patient, when the computer instructions are carried out in a computer. The technical effects necessary according to the invention can thus be realized on the basis of the instructions of the computer program in accordance with the invention. Such a computer program may be stored on a carrier or it may be available over the internet or some other computer network. Prior to its execution the computer program is loaded into a computer in that it is read from the carrier, for example by means of a CD-ROM player, or from the internet and is stored in the memory of the computer. The computer includes inter alia a central processor unit (CPU), a bus system, memory means, e.g. a RAM or ROM, etc., and input/output units.

A core idea of the invention is to utilize data representing disturbances of the rest-activity rhythm of a patient, especially disturbances of a patient's sleep, as an indicator of depression. According to the invention, an actimetric technique is used to provide an objective measure for depression and/or a relapse into depression.

Depression or a relapse into depression may be diagnosed, for example, if the measured activity pattern and the deviations from the known activity profile indicate a sleep disorder of the patient during a time period of at least one or two weeks. In order to determine whether or not a depression or a relapse into depression exists, one or more of the following parameters are obtained and analyzed: time in bed, total sleep time, sleep period time, sleep efficiency index, number of changes between sleep stages, number of awakenings or arousals, sleep onset latency, deep sleep latency, REM latency, number and duration of sleep cycles (one cycle=start non-REM until end REM sleep), REM density, and other factors. Besides sleep disorder, another indication for a depression or a relapse into depression which can be used with the present invention is a considerably reduced day-time activity of the patient. If one or more further indications are detected additionally, e.g. a characteristic variation of the patient's heart rate, the likelihood of a depression or a relapse into depression is high.

The present invention provides an objective measure for a depression as well as for a relapse into depression. Since the result of the examination is obtained automatically, the ease of use for the patient is greatly enhanced. The automatically obtained results can be used as a high-quality basis for a subsequent medical interview and treatment. A major advantage of the present invention is the early moment of diagnosis compared with the known interview techniques. The present invention may be applied for screening for symptoms of depression in high-risk patients such as patients who already had an episode of depression (relapse detection), or post-MI patients, or patients with heart insufficiency who are at a higher risk of suffering depression than an unselected population and who are probably already closely monitored because of their cardiac disease.

With the present invention the patient can be monitored continuously in a cost-effective manner over several days to several months. The method is easy to implement and can be used in the patient's home without additional stress caused by external observation. Furthermore, the patient is not continuously aware of being diagnosed, which reduces the stress further. With the invention, the number of clinical stays in the ambulatory sector or in the general practitioner's environment can be markedly reduced. Finally, an early diagnosis of depression and of relapses into depression permits a more effective treatment, leads to less hospitalisations, fewer days of inability to work, and consequently to cost reductions.

These and other aspects of the invention will be further elaborated on the basis of the following embodiments, which are defined in the dependent claims.

In a preferred embodiment, the determination of depression is based on a comparison between the measured patient's activity and a known activity profile. The known activity profile may be a profile recorded by means of the depression detection system or some other medical system at an earlier point in time (e.g. one year before the monitoring is carried out), or the known activity profile may have been composed at an earlier point in time on the basis of a standard profile of a healthy patient. Alternatively, the known activity profile is obtained with the depression detection system during the actual monitoring. It is assumed in that case that the patient using the depression detecting system shows a normal (non-depressive) activity profile at least during a period of several days or weeks. The activity profile is used as the known activity profile by the signal-processing unit in order to find deviations between the known (normal) activity profile and the current activity of the patient. In other words, the present invention is based on the detection of differences between activity profiles.

In another preferred embodiment, the determination of depression is based on a statistical analysis of the measured patient's activity. For example, the average or standard deviation of activity parameters are determined every hour over a certain period of time. The statistical analysis may be used in addition to the comparison-based technique.

In still another embodiment of the invention, the determination of depression is based on a frequency analysis. Here the signals from the accelerometer are analyzed over specific periods of time, e.g. 1 hour, 1 night, 24 hours, etc., by means of a fast Fourier transformation technique. The frequency content of the activity signal and/or the power density of the spectrum changes in the case of relapse into depression. In yet another embodiment, the determination of depression is based on a wavelet analysis. Whereas Fourier analysis consists of splitting up of the signal into sine waves of different frequencies, wavelet analysis is the breaking up of a signal into shifted and scaled versions of the original (or mother) wavelet. Wavelets are mathematical functions of limited duration that have an average value of zero. They tend to have an irregular and asymmetrical waveform. Wavelet analysis has advantages over Fourier analysis when the signal contains discontinuities, sharp changes, or trends. In contrast to Fourier analysis, wavelet analysis is capable of detecting the exact location in time of a discontinuity.

An accelerometer is preferably used for sensing the patient's activity. The use of accelerometers is especially advantageous, because they are small, easy to use, available with one to three sensing axes, and cheap. An accelerometer with two or three sensing axes is preferably used in order to obtain information about the position of the patient's body during a certain period of time. This information is preferably used by the signal-processing unit in order to analyze the measured activity of the patient in relation to the known activity profile.

Accelerometers can be easily integrated into small and convenient patient devices or even integrated into his clothing. Any kind of accelerometer may be used, such as pendulous accelerometers, vibrational accelerometers, or electromagnetic accelerometers. The sensors can be realized as wrist-worn, ankle-worn, or torso-worn devices. Alternatively, the sensors may be integrated into the patient's clothing, e.g. underwear, long sleeves of shirts, socks, or in a belt. Another alternative is to implant the sensors, e.g. under the patient's skin.

In a further embodiment of the invention, all patient-based parts of the depression detection system are integrated into a single device. This further improves the ease of use. Parts of the depression detection system, especially the signal-processing unit, may be realized in hardware, e.g. a data processor or the like, or as a computer program designed for carrying out data processing, or a combination of the two.

The interface unit preferably comprises a display, e.g. a light-emitting diode (LED), that switches on when a depression is detected. In another embodiment of the invention, the user interface comprises a wireless data communication system, e.g. adapted to establish a communication link to a personal computer, a mobile phone, or a personal digital assistant (PDA), etc. If a psychometric questionnaire, e.g. Beck's Depression inventory or the WHO-5 questionnaire, is electronically implemented on such a computer, mobile phone, or PDA, additional information about the patient obtained by the depression detection system according to the present invention can be used to support a questionnaire-based decision as to whether a relapse into depression is present or not. In still another embodiment, the data communication unit of the depression detection system is adapted to send a message, e.g. an E-mail or SMS, to the patient's general practitioner or psychiatrist, if e.g. profiles indicating depression are repeatedly detected.

In still another embodiment of the invention, data from a patient device is sent by means of the interface unit via a data communication link, e.g. via a mobile phone or via the internet, to an external computer, e.g. to a server. At least part of data processing usually carried out by the signal-processing unit is carried out on the server. The server sends a regular, for example daily feedback to the patient (e.g. on the patient's mobile phone), and also sends feedback (e.g. via E-mail) to the patient's general practitioner or psychiatrist, if necessary. With this embodiment the patient-based device, especially the signal-processing unit, can be designed to be less complex, since part of the data processing is carried out in the external server or equivalent device.

In another embodiment of the invention, the depression detection system further comprises an electrocardiogram (ECG) sensor unit and/or an electromyogram (EMG) sensor unit and/or an electrooculogram (EOG) sensor unit, while the signal-processing unit is adapted to produce the result using these additional patient-related data. An ECG signal, e.g. a heart rate or heart rate variability, may be used to improve the distinction between wakefulness and sleep with respect to the activity measurement alone. Furthermore, ECG signals yield some parameters characteristic of depression such as an elevated resting heart rate, decreased heart rate variability, and exaggerated heart rate response to an orthostatic challenge. EMG and EOG signals yield some rapid eye movement (REM) sleep parameters characteristic of depression, such as a shortened REM sleep latency, a higher amount of REM sleep per night than in healthy individuals, and an altered distribution of REM sleep during the night (REM sleep shifted from the second to the first half of the night). Preferably, the depression detection system is adapted to use such ECG, EMG, and/or EOG signals as a data input for the signal-processing unit in order to provide a more precise result. For this purpose the signal processing unit includes an interface for data transfer from ECG, EMG, and/or EOG sensor units. Alternatively, the depression detection system according to the present invention is partly or fully integrated into an ECG, EMG, or EOG system.

In still another embodiment, the patient's body temperature is obtained by a suitable measuring device and used as an input signal for the signal-processing device in order to provide additional information about the patient's circadian rhythm. Because of the minimal thermal fluctuation, the temperature is preferably measured near the patient's torso.

In another embodiment of the invention, the cortisol level of the patient is observed. Cortisol is a steroid hormone. In a large number of cases patients suffering from depressions show an increased cortisol level. The cortisol level test is preferably carried out by an internal sensor unit or an external sensor unit that can be connected to the depression detection system. The results obtained in the cortisol level test are transferred to the signal-processing unit, which is adapted to employ these results in order to detect depression.

In yet another embodiment of the invention, speech characteristics of the patient are used as an additional indication for the existence of depression. The patient's speech is analyzed by an internal sensor unit or an external sensor unit that can be connected to the depression detection system, wherein the acoustic properties (e.g. fundamental frequency, energy, speaking rate, amplitude modulation, formants, power distribution, and other factors) of the speech are used as indicators for depression. The results obtained by the sensor unit are transferred to the signal-processing unit. The signal-processing unit is adapted to process these results as an additional input parameter.

These and other aspects of the invention will be described in detail hereinafter, by way of example, with reference to the following embodiments and the accompanying drawings, in which:

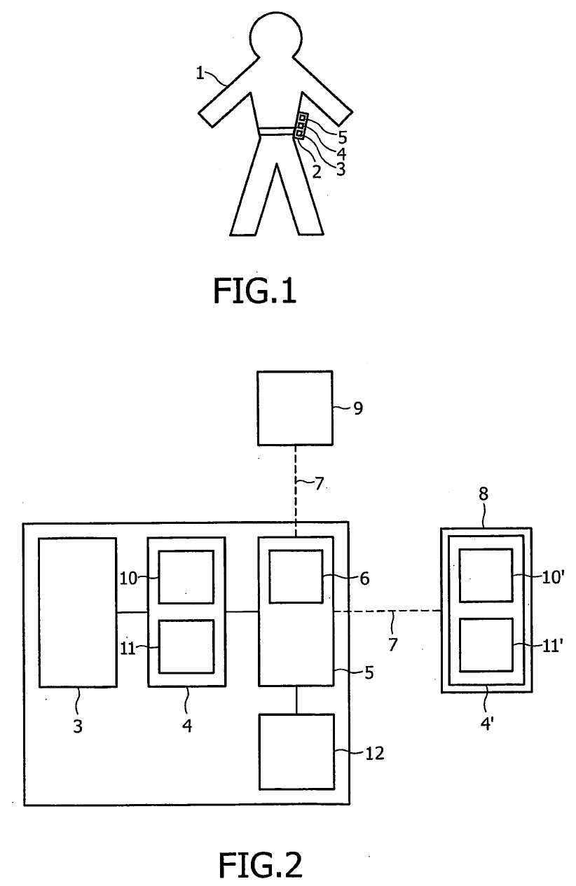

FIG. 1 is a schematic picture of a patient using the system according to the invention;

FIG. 2 is a block diagram showing the system according to the invention.

FIG. 1 illustrates a patient 1 monitored by a depression detection system according to the invention. The depression detection system is implemented as a patient-based, body-mounted device 2 and includes an accelerometer 3 with three sensing-axes to measure the patient's activity, a signal-processing unit 4 to produce a result based on a comparison between the measured patient's activity and a known activity profile, and an interface unit 5 adapted to provide a signal to the patient in dependence on the result of the comparison. The patient-based device 2 is integrated into the patient's underwear and located near the patient's torso. Compared with another position, e.g. on the patient's wrist, the amount of insignificant activity data caused by nonrelevant arm movements is reduced. The patient-based device 2 is shown strongly enlarged for the purpose of illustration.

As shown in FIG. 2, the accelerometer 3 is connected to the signal-processing unit 4, and the signal-processing unit 4 is connected to the user interface 5, which comprises a data communication system 6 adapted to establish a wireless data communication link 7 to an external personal computer 8 using the Bluetooth-communication protocol.

The accelerometer 3 measures the patient's activity over a longer period of time, e.g. several months. Furthermore, the patient's heart rate is obtained by an external ECG sensor unit 9. The accelerometer signals and the ECG signals are transferred to the signal processing unit 4, where they are stored in a data storage device 10. Subsequently a number of activity profiles are generated within the signal-processing unit 4 by a dedicated computer program carried out on a data processor 11. For example, an activity profile is generated by the data processor 11 on a daily basis, taking into account e.g. the duration of rest periods, sleep characteristics such as the duration of REM phases, and other activity data. Each activity profile is stored in the data storage device 10.

After predetermined or automatically adjusted time intervals, e.g. once a week, the current activity profile is compared with a single prior activity profile or a number of prior activity profiles in order that the signal processing unit 4 can determine deviations in the patient's activity. If no such deviations are detected or if the deviations are below a certain threshold, a “no depression” result is generated. The threshold may be predetermined or automatically adjusted according to prior activity profiles and/or other parameters. Preferably, the currently valid threshold is stored in the data storage device 10. The “no depression” signal is then transmitted by the interface unit 5 to an integrated display unit 12, e.g. switching on a green LED. If deviations are determined which exceed the threshold, a “depression” result is generated and transferred to the user interface 5. Subsequently the “depression” signal is transmitted by the interface unit 5 to the integrated display unit 12, e.g. switching on a red LED. A mobile phone, PDA, or some other external display unit using a dedicated depression-displaying software may be used instead of an internal LED display.

In another embodiment of the invention, a signal-processing unit 4′ is provided externally, e.g. as part of the external personal computer 8. In this case the interface unit 5 is adapted to transfer the input accelerometer signals and other input signals to the external signal-processing unit 4′ via a wireless data communication link 7. Measuring data, activity profiles, and further subsequent results are processed and stored externally by means of the external signal-processing unit 4′ and an external storage unit 10′. In other words, the patient-based device 2 merely comprises the accelerometer 3 and the interface unit 5 and can therefore be designed in a much smaller and more convenient way. Furthermore, the energy consumption is considerably reduced, leading to an extended operating time of the patient-based device 2. The results of the depression detection are provided to the patient from the external signal processing unit 4′ e.g. by means of a display.

It will be evident to those skilled in the art that the invention is not limited to the details of the foregoing illustrative embodiments, and that the present invention may be embodied in other specific forms without departing from the spirit or essential attributes thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein. It will furthermore be evident that the word “comprising” does not exclude other elements or steps, that the words “a” and “an” do not exclude a plurality, and that a single element, such as a computer system or another unit, may fulfill the functions of several means recited in the claims. Any reference signs in the claims shall not be construed as limiting the claim concerned.

### REFERENCE LIST

1 patient
2 patient-based device
3 actimetric sensor unit
4 signal processing unit
5 user interface
6 data communication system
7 data communication link
8 external personal computer
9 ECG sensor unit
10 data storage device
11 data processor
12 display unit

1. A depression detection system, the system comprising
a patient-based device (2) comprising an actimetric sensor unit (3) adapted to measure a patient's activity and
a signal-processing unit (4, 4′) adapted to obtain a depression detection result based on the measured activity of the patient.

2. The system (2) as claimed in claim 1, wherein the signal-processing unit (4, 4′) is adapted to obtain the depression detection result from a comparison between the measured patient's activity and an activity profile.

3. The system (2) as claimed in claim 1, wherein the signal processing unit (4, 4′) is adapted to obtain the depression detection result from a statistical analysis of the measured patient's activity.

4. The system (2) as claimed in claim 1, wherein the patient-based device (2) further comprises an interface unit (5) adapted to transfer measuring data to an external signal-processing unit (4′) via a data communication link (7).

5. The system (2) as claimed in claim 1, wherein the signal-processing unit (4) is part of the patient-based device (2), and the interface unit (5) is adapted to provide a signal to the patient in dependence on the patient's activity.

6. The system (2) as claimed in claim 1, wherein the patient-based device (2) further comprises a display unit (12) for providing a signal to the patient in dependence on the depression detection result.

7. The system (2) as claimed in claim 1, wherein the actimetric sensor unit (3) comprises an accelerometer.

8. The system (2) as claimed in claim 7, wherein the accelerometer is adapted to obtain information about the spatial position of the patient's body.

9. The system (2) as claimed in claim 1, wherein the actimetric sensor unit (3) is implemented in a wrist-worn, ankle-worn, or torso-worn device.

10. The system (2) as claimed in claim 1, wherein the actimetric sensor unit (3) is integrated into the patient's clothing.

11. The system (2) as claimed in claim 1, wherein the signal-processing unit (4) is adapted to produce the depression detection result using additional patient-related data.

12. The system (2) as claimed in claim 1, further comprising an ECG sensor unit (9) and/or an EMG sensor unit and/or an EOG sensor unit.

13. A method of detecting depression, the method comprising the steps of:
measuring a patient's activity with an actimetric sensor unit (3), and
obtaining a depression detection result on the basis of the measured activity of the patient.

14. A computer program comprising computer instructions for measuring a patient's activity by means of an actimetric sensor unit (3) and computer instructions for obtaining a depression detection result on the basis of the measured activity of the patient when said computer instructions are carried out in a computer (11).
